(19) United States
(12) Patent Application Publication
(10) Pub. No.: US 2012/0289867 A1
(43) Pub. Date: Nov. 15, 2012

(54) STATE DETERMINING DEVICE AND STATE DETERMINATION METHOD

(75) Inventor: KOUICHIROU KASAMA, Kawasaki-shi (JP)

(73) Assignee: FUJITSU LIMITED, Kawasaki-shi (JP)

(21) Appl. No.: 13/462,703

(22) Filed: May 2, 2012

(30) Foreign Application Priority Data
May 12, 2011 (JP) 2011-107685

(51) Int. Cl. A61B 5/11

(52) U.S. Cl. 600/595

(57) ABSTRACT

A state determining device includes a sensor and a processor. The sensor of the state determining device detects body motions of a test subject without physical contact. The processor of the state determining device determines whether the test subject is asleep or awake based on numbers of body motions detected for predetermined time periods.

### CROSS-REFERENCE TO RELATED APPLICATION

This application is based upon and claims the benefit of priority of the prior Japanese Patent Application No. 2011-107685, filed on May 12, 2011, the entire contents of which are incorporated herein by reference.

### FIELD

The embodiment discussed herein is related to a state determining device and a state determination method.

### BACKGROUND

There is a technique for detecting a motion (referred to as a body motion) of a body of a test subject who is in bed and determining a state of the test subject on the basis of a result of the detection. As a device that uses the technique, there is an actigraph that uses an acceleration sensor and determines whether or not a test subject is awake or asleep. The actigraph is an activity meter that measures the amount of activity of a body. When a test subject goes to bed while wearing a wrist type actigraph having an acceleration sensor on his/her wrist, the actigraph records values output from the acceleration sensor at certain time intervals (for example, 1 minute). The actigraph uses the values output from the acceleration sensor and the amount of a change of the values for a certain time period to detect a body motion of the test subject who is in bed. Then, the actigraph determines whether or not the test subject is asleep on the basis of a result of the detection.

Japanese Laid-open Patent Publications Nos. 2011-24656 and 05-212003 and International Publication Pamphlets Nos. WO 2004/078132, WO 2000/026841, WO 2002/073342, and WO 2002/073343 disclose related techniques.

According to the aforementioned technique, however, since the test subject goes to bed while wearing the device on the body, the test subject may feel that the device is inconvenient and uncomfortable, depending on the posture and environment of the test subject who is in bed. As a matter of course, when the test subject is asleep and the device is detached from the test subject or removed by himself/herself for some reason, a body motion of the test subject is not detected by the device. In addition, when the test subject is in bed, motions of the body vary. When the acceleration sensor is used to detect the motions of the body, it is difficult to detect a quasi-static motion (such as a uniform motion) without acceleration. This reduces accuracy of determining the state of the test subject on the basis of a result of detecting a body motion.

### SUMMARY

According to an aspect of the present invention, provided is a state determining device including a sensor and a processor. The sensor detects body motions of a test subject without physical contact. The processor determines whether the test subject is asleep or awake based on numbers of body motions detected for predetermined time periods.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating a functional configuration of a state determining device;

FIG. 2 is a diagram illustrating a hardware configuration of a state determining device;

FIG. 3 is a flowchart describing operations of a state determining device;

FIG. 4 is a flowchart describing a process of detecting a body motion using an ultrasonic sensor;

FIG. 5 is a flowchart describing a process of detecting a body motion using a pyroelectric infrared sensor;

FIG. 6 is a diagram illustrating exemplary adjustment coefficients that are used to calculate a state determination value; and

FIG. 7 is a diagram illustrating an example of data that is stored in a determination result correction table.

### DESCRIPTION OF EMBODIMENT

Hereinafter, an embodiment of a state determining device and a state determination method is described in detail with reference to the accompanying drawings. However, the state determining device and the state determination method disclosed herein are not limited by the embodiment.

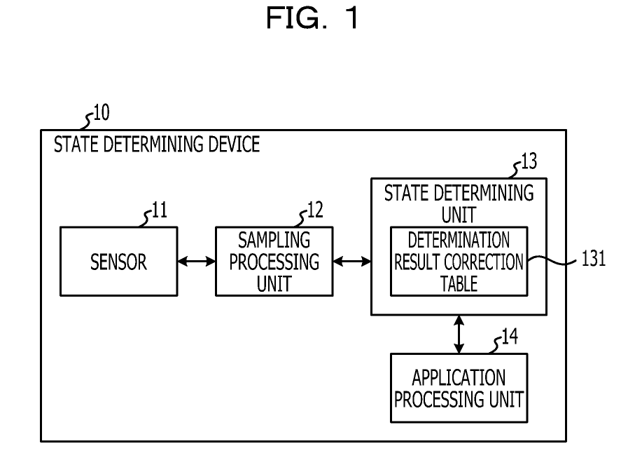

The embodiment of the state determining device is described with reference to the accompanying drawings. First, the configuration of a state determining device 10 according to the embodiment is described below. FIG. 1 is a diagram illustrating a functional configuration of the state determining device 10 according to the embodiment. As illustrated in FIG. 1, the state determining device 10 includes a sensor 11, a sampling processing unit 12, a state determining unit 13 and an application processing unit 14. The sensor 11, the sampling processing unit 12, the state determining unit 13 and the application processing unit 14 are connected to each other so that a signal and data may be unidirectionally or bidirectionally input and output therebetween.

The sensor 11 is a noncontact sensor that detects a body motion of a test subject who is in bed. Upon detecting a body motion of the test subject, the sensor 11 transmits a body motion detection signal to the sampling processing unit 12. The sensor 11 is an ultrasonic sensor, for example. An ultrasonic sensor periodically transmits an ultrasonic wave having a certain frequency for certain pulses and receives an ultrasonic wave reflected by an object. The ultrasonic sensor compares the intensity (amplitude) of a received ultrasonic wave for a current pulse with the intensity (amplitude) of a received ultrasonic wave for a previous pulse. When the intensities are different from each other, the ultrasonic sensor transmits, to the sampling processing unit 12, a notification of detecting a body motion. In order to accurately sense a body motion, it is preferable that the frequency of the ultrasonic wave transmitted by the ultrasonic sensor be a frequency (of, for example, approximately 10 KHz to 50 KHz) that enables the ultrasonic wave to pass through a blanket and clothes and to be reflected by the body of the test subject. In addition, the sensor 11 may be a pyroelectric infrared sensor that may be installed in a home electrical appliance or the like. The pyroelectric infrared sensor uses a pyroelectric effect, detects an infrared ray emitted by the test subject and senses an initial body motion of the test subject.

The sampling processing unit 12 receives the body motion detection signal from the sensor 11 and records the reception time at which the sampling processing unit 12 receives the body motion detection signal.

The state determining unit 13 calculates the number of motions of the body detected for a certain time period on the basis of information on the reception time of the body motion detection signal. On the basis of the result of the calculation, the state determining unit 13 makes a state determination, that is, the state determining unit 13 determines whether the state of the test subject is a sleeping state or an awake state. Specifically, the state determining unit 13 calculates a state determination value M using the numbers of body motions detected for certain time periods before and after a certain time for which the state of the body is to be determined. For example, the certain time period before the certain time is 4 minutes, and the certain time period after the certain time is 3 minutes. When the state determination value M is equal to or larger than a threshold T, the state determining unit 13 determines that the test subject is in the awake state. When the state determination value M is smaller than the threshold T, the state determining unit 13 determines that the test subject is in the sleeping state. The state determining unit 13 transmits, to the application processing unit 14, a notification indicating the result (hereinafter, referred to as a state determination result) of determining the state of the test subject. The state determining unit 13 has a determination result correction table 131. When the state determination result is estimated to be incorrect, the state determining unit 13 references the determination result correction table 131 and corrects the state determination result. The process of correcting the state determination result is described later in detail.

The application processing unit 14 causes a display unit to display information on the sleeping or awake state indicated by the notification transmitted from the state determining unit 13.

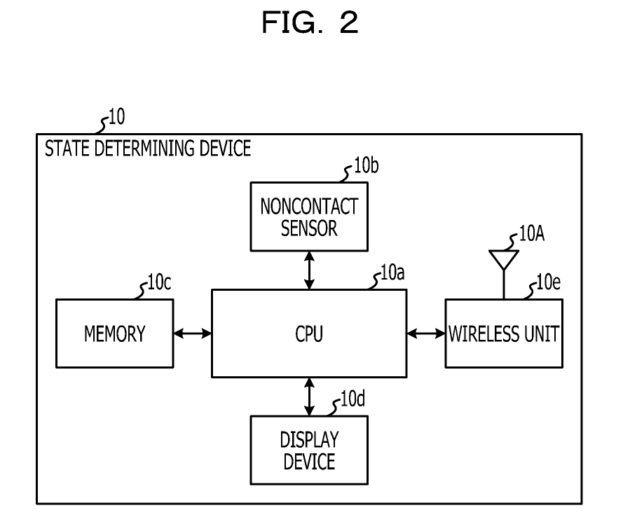

The state determining device 10 may be a mobile phone, for example. FIG. 2 is a diagram illustrating a hardware configuration of a mobile phone as the state determining device 10. As illustrated in FIG. 2, the state determining device 10 physically includes a central processing unit (CPU) 10a, a noncontact sensor 10b, a memory 10c, a display device 10d, and a wireless unit 10e that has an antenna 10A. As described above, the sensor 11 is achieved by the noncontact sensor 10b that is the ultrasonic sensor or the pyroelectric infrared sensor. The sampling processing unit 12, the state determining unit 13 and the application processing unit 14 are achieved by an integrated circuit such as the CPU 10a. Information on the reception time of the body motion detection signal is stored in the memory 10c that is a random access memory (RAM), a flash memory or the like. The state determination result that indicates the sleeping state or the awake state is displayed on the display device 10d that is a liquid crystal display (LCD) or the like.

Next, operations of the state determining device 10 are described. The operations are described based on the assumption that the state determining device 10 is arranged at, for example, a location near the head of the test subject who is in bed so that the sensor 11 may detect a body motion of the test subject.

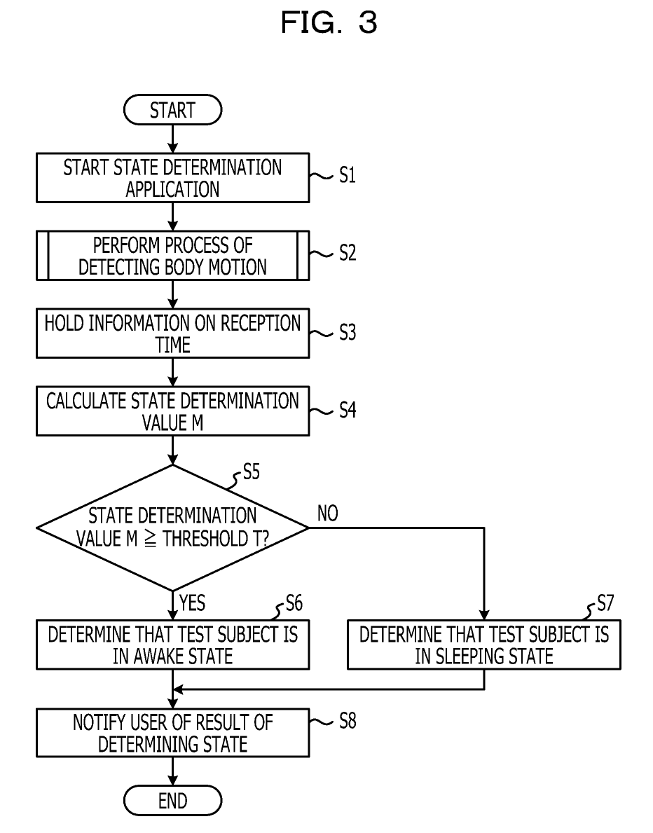

FIG. 3 is a flowchart describing the operations of the state determining device 10. When a user starts a state determination application of the state determining device 10 (S1), the state determining device 10 starts to perform a process of detecting a body motion (S2).

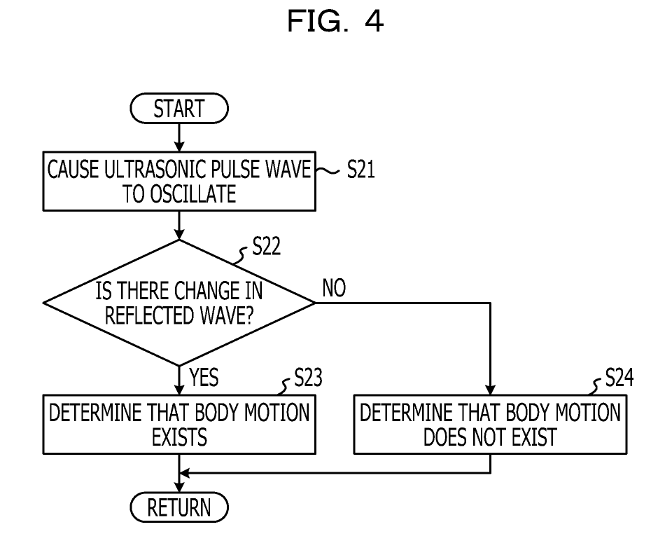

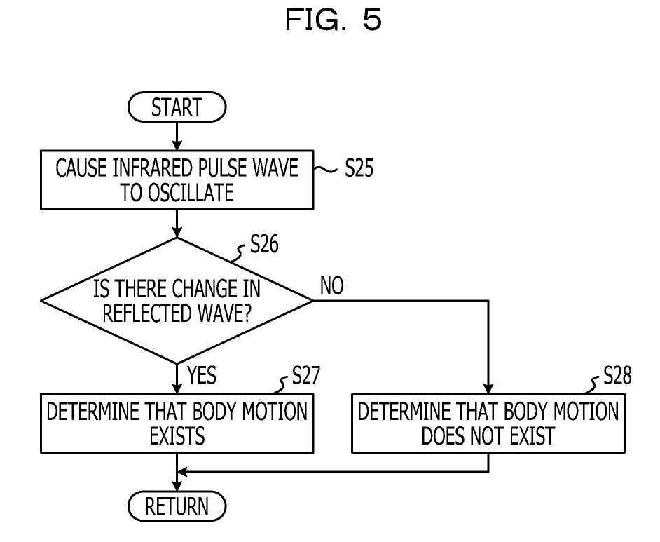

The process of detecting a body motion is described with reference to FIGS. 4 and 5. FIG. 4 is a flowchart describing the process of detecting a body motion using an ultrasonic sensor. When the process of detecting a body motion starts, the ultrasonic sensor as the sensor 11 transmits an ultrasonic pulse wave at time intervals of 50 ms (S21). The ultrasonic sensor monitors the arrival time and the amplitude of a reflected wave of the ultrasonic pulse wave to determine whether or not the arrival time or the amplitude changes (S22). When the arrival time or the amplitude of the reflected wave changes as a result of monitoring (Yes in S22), the ultrasonic sensor determines that a body motion exists (S23). On the other hand, when both of the arrival time and the amplitude of the reflected wave do not change (No in S22), the ultrasonic sensor determines that a body motion does not exist (S24).

When the state determining device 10 detects a body motion using the pyroelectric infrared sensor, the state determining device 10 performs a similar process to the process that is performed using the ultrasonic sensor. FIG. 5 is a flowchart describing the process of detecting a body motion using the pyroelectric infrared sensor. As illustrated in FIG. 5, the process of detecting a body motion using the pyroelectric infrared sensor is performed in the same manner as the process of detecting a body motion illustrated in FIG. 4, except that the pyroelectric infrared sensor transmits an infrared pulse wave at time intervals of 50 ms (S25 illustrated in FIG. 5). Thus, a detailed description of the process of detecting a body motion using the pyroelectric infrared sensor is omitted. S25 to S28 illustrated in FIG. 5 correspond to S21 to S24 illustrated in FIG. 4, respectively.

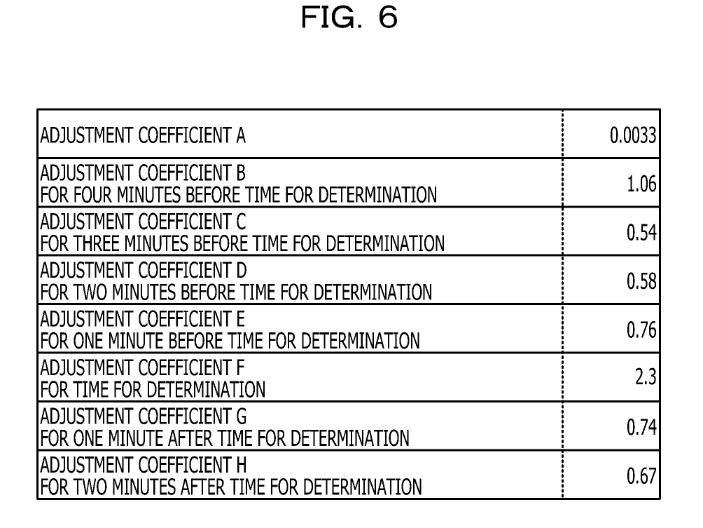

Returning to FIG. 3, when the process of detecting a body motion is terminated, the state determining device 10 records information on the time when the sampling processing unit 12 receives the body motion detection signal (S3). In S4, the state determining device 10 calculates the state determination value M on the basis of results of detections of body motions corresponding to the respective times indicated by the information recorded in S3. The state determination value M is an index of the determination of whether the state of the test subject is the awake state or the sleeping state. A method for calculating the state determination value M is described below with reference to FIG. 6. The time for which the state of the test subject is determined is treated as a reference time. The number of body motions detected for a one-minute time period, from a time four minutes before the reference time to a time three minutes before the reference time, is indicated by “Mb”. The numbers of body motions that are detected over time are described below. The number of body motions detected for a one-minute time period, from the time three minutes before the reference time to a time two minutes before the reference time, is indicated by “Mc”. The number of body motions detected for a one-minute time period, from the time two minutes before the reference time to a time one minute before the reference time, is indicated by “Md”. The number of body motions detected for a one-minute time period, from the time one minute before the reference time to the reference time, is indicated by “Me”. The number of body motions detected for a one-minute period, from the reference time to a time one minute after the reference time, is indicated by “Mf”. The number of body motions detected for a one-minute time period, from the time one minute after the reference time to a time two minutes after the reference time, is indicated by “Mg”. The number of body motions detected for a one-minute time period, from the time two minutes after the reference time to a time three minutes after the reference time, is indicated by “Mh”. As illustrated in FIG. 6, an adjustment coefficient that is multiplied to the total of the numbers of the detected body motions is indicated by “A”. Adjustment coefficients that are multiplied to the numbers of the body motions detected for the aforementioned one-minute time periods are indicated by “B”, “C”, “D”, “E”, “F”, “G” and “H”, as illustrated in FIG. 6. The state determination value M is calculated according to the following equation. The state determination value M=A*(BMb+C*Md+D*Md+E*Me+F*Mf+G*Mg+H*Mh). The adjustment coefficients may be set to arbitrary values and changed. In the state determining unit 13, the adjustment coefficient A is set to 0.0033, the adjustment coefficient B is set to 1.06, the adjustment coefficient C is set to 0.54, the adjustment coefficient D is set to 0.58, the adjustment coefficient E is set to 0.76, the adjustment coefficient F is set to 2.3, the adjustment coefficient G is set to 0.74, and the adjustment coefficient H is set to 0.67, in advance, for example.

Returning to FIG. 3, in S5, the state determining device 10 compares the state determination value M calculated in S4 with the threshold T and determines whether or not the state determination value M is equal to or larger than the threshold T. The threshold T may be set to an arbitrary value and changed. The threshold T is 1.0, for example. When T is 1.0 and M≧1.0 (Yes in S5), the state determining device 10 determines that the test subject is in the “awake state” (S6). On the other hand, when M<1.0 (No in S5), the state determining device 10 determines that the test subject is in the “sleeping state” (S7). The state determining device 10 notifies the user of the state determination result through the display device 10d (S8).

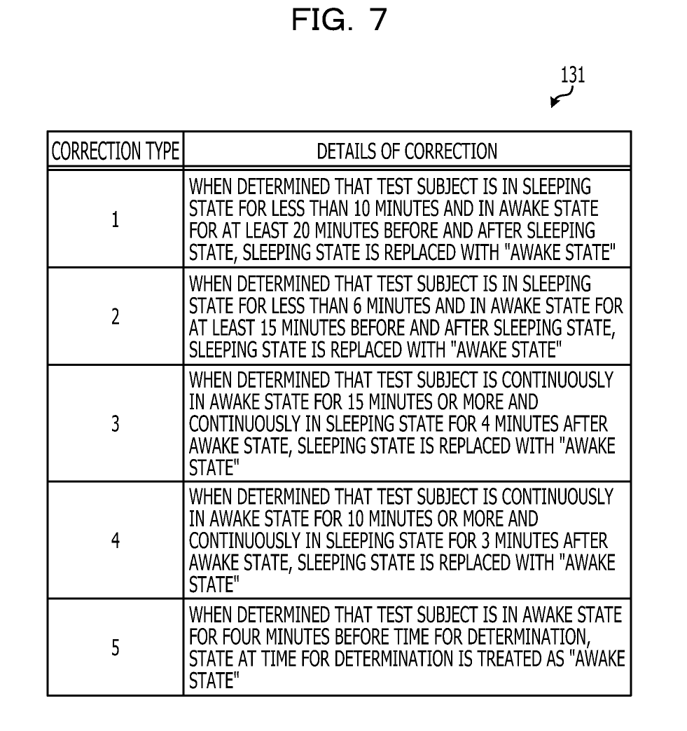

The state determined in S7 may be corrected on the basis of the previous and next state determination results. Specifically, when the state determination result is estimated to be incorrect, the state determining unit 13 may reference the determination result correction table 131 and correct the state determination result. FIG. 7 is a diagram illustrating an example of data that is stored in the determination result correction table 131. First, details of a correction of a first correction type are described below. The first correction type indicates that when the state determining unit 13 determines that the test subject is in the sleeping state for less than 10 minutes and in the awake state for at least 20 minutes before and after the sleeping state, the sleeping state may be estimated to be actually the awake state. Therefore, the state determining device 10 replaces the “sleeping state” with the “awake state”. Then, the correction process is terminated. On the other hand, when any of the requirements that are indicated by the first correction type is not satisfied, that is, when the test subject is in the sleeping state for 10 minutes or more or when the test subject is in the awake state for less than 20 minutes before or after the sleeping state, the state determining device 10 does not perform the correction of the first correction type and tries to perform a correction of a second correction type described below.

The second correction type indicates that when the state determining unit 13 determines that the test subject is in the sleeping state for less than 6 minutes and in the awake state for at least 15 minutes before and after the sleeping state, the sleeping state may be estimated to be actually the awake state and the state determining device 10 replaces the “sleeping state” with the “awake state”. Then, the correction process is terminated. On the other hand, when any of the requirements that are indicated by the second correction type is not satisfied, that is, when the test subject is in the sleeping state for 6 minutes or more or when the test subject is in the awake state for less than 15 minutes before or after the sleeping state, the state determining device 10 does not perform the correction of the second correction type and tries to perform a correction of a third correction type described below.

The third correction type indicates that when the state determining unit 13 determines that the test subject is continuously in the awake state for 15 minutes or more and continuously in the sleeping state for 4 minutes after the awake state, the state determining device 10 replaces the “sleeping state” with the “awake state”. Then, the correction process is terminated. On the other hand, when any of the requirements that are indicated by the third correction type is not satisfied, that is, when the test subject is continuously in the awake state for less than 15 minutes or when the test subject is continuously in the sleeping state for less than 4 minutes after the awake state, the state determining device 10 does not perform the correction of the third correction type and tries to perform a correction of a fourth correction type described below.

The fourth correction type indicates that when the state determining unit 13 determines that the test subject is continuously in the awake state for 10 minutes or more and continuously in the sleeping state for 3 minutes after the awake state, the state determining device 10 replaces the “sleeping state” with the “awake state”. Then, the correction process is terminated. On the other hand, when any of the requirements that are indicated by the fourth correction type is not satisfied, that is, when the test subject is continuously in the awake state for less than 10 minutes or when the test subject is continuously in the sleeping state for less than 3 minutes after the awake state, the state determining device 10 does not perform the correction of the fourth correction type and tries to perform a correction of a fifth correction type described below.

The fifth correction type indicates that when the state determining unit 13 determines that the test subject is in the awake state at the time four minutes before the reference time, the state determining device 10 treats the state of the test subject at the reference time as the “awake state”. Then, the correction process is terminated. On the other hand, when the requirement that is indicated by the fifth correction type is not satisfied, that is, when the requirements that are indicated by the five correction types are not satisfied, the state determining device 10 does not correct the state determination result and terminates the correction process.

In the process of determining a state on the basis of a result of detecting a body motion, the state determining device 10 may determine a state that is different from the actual state, depending on the adjustment coefficients A to H and the threshold T. This is due to the fact that the sensor 11 may incorrectly detect a body motion or there are differences among body motion patterns of persons. For example, when a body motion sensing capability of the sensor 11 is low, or when a frequency of motions of the body of the test subject who is in the awake state is low, the state determining device 10 may determine that the test subject is in the sleeping state regardless of the fact that the test subject is actually in the awake state. Thus, the state determining device 10 performs the aforementioned correction process on the basis of characteristics that are generally included in state transitions relating to the sleeping state and the awake state. Therefore, even when the aforementioned incorrect determination is made, the determination result may be corrected. Thus, the state determining device 10 may maintain a high-level state determination result obtained from the test subject and inform the user of accurate information. As a result, the reliability of the state determining device 10 is improved.

As described above, the state determining device 10 according to the present embodiment includes the sensor 11 and the state determining unit 13. The sensor 11 detects a body motion of the test subject who is in bed without contacting the test subject. The state determining unit 13 uses the number of body motions detected for a certain time period and determines whether the test subject is in the awake state or the sleeping state. The state determining unit 13 treats, as the reference time, the time for which the state of the test subject is determined. The state determining unit 13 uses the number of body motions detected for a certain time (for example, 4 minutes) before the reference time and the number of body motions detected for a certain time (for example, 3 minutes) after the reference time, and calculates the state determination value M that is the index of the determination of whether the state of the test subject is the awake state or the sleeping state. In addition, the state determining unit 13 determines, on the basis of the result of the comparison of the state determination value M with the threshold T, whether the test subject is in the sleeping state or the awake state. The state determining device 10 uses the noncontact sensor to detect a body motion of the test subject. Thus, the state determining device 10 may detect a very small body motion of the test subject and a quasi-static body motion without acceleration, which is not detected by an acceleration sensor. Therefore, the state determining device 10 may accurately determine the state (awake state or sleeping state) of the test subject on the basis of a change in the number of detected body motions.

The state determining device 10 includes the sensor 11 that is the ultrasonic sensor or the pyroelectric infrared sensor. The ultrasonic sensor and the pyroelectric infrared sensor are noncontact sensors. Thus, the user places the state determining device 10 near the user while in bed without wearing the state determining device 10, and may recognize easily and quickly a result of determining the state of the user. Thus, the user does not feel that the state determining device 10 is inconvenient and uncomfortable, compared with an acceleration sensor that is included in an actigraph. In addition, an inconvenience, such that the state of the test subject is not determined owing to a removal of the state determining device 10 from the body of the test subject who is in bed by accident or by himself/herself for some reason, may be avoided. In addition, the noncontact sensor may detect a very small motion and uniform body motion of the test subject who is asleep, although the acceleration sensor does not detect the very small motion and uniform motion. Thus, the state determining device 10 uses the result (number of detected body motions) detected by the noncontact sensor in order to determine the state of the test subject, and may determine the state of the test subject on the basis of the accurate result of the detection, compared with a process of determining the state of the test subject using the acceleration sensor. As a result, the state determining device 10 may accurately determine the state of the test subject.

Next, an application example of the state determining device 10 is described.

For example, the state determining device 10 calculates a sleeping time of the user on the basis of the result of the determination of whether or not the user that is the test subject is in the awake state, and estimates the quality of the sleep on the basis of the sleeping time. When the state determining device 10 estimates that the quality of the sleep of the user is poor, the state determining device 10 may advise the user to increase the amount of an activity during daytime. There is a research report indicating that the shorter sleeping times, the lower the amount of consumed calories and the amount of consumed body fat and the higher a risk of obesity. Thus, the state determining device 10 may provide, to a user whose sleeping time calculated by the state determining device 10 is short, a guidance that indicates an effective diet method. In addition, the state determining device 10 may advise, on the basis of the sleeping time calculated from the result of determining the state of the user, the user to do aerobic exercises, soundly sleep, and alleviate mental and physical fatigue. In addition, the result of the determination that is performed by the state determining device 10 may be used for various purposes such as an effective skin improvement method, a stress release method and a proposal of healthcare. Therefore, the state determining device 10 references a result of determining the state, detects sleeping habits of the test subject on the basis of temporal records of the awake and sleeping states, and may thereby instruct the user to habitually act according to bedtime, a wake-up time and a sleeping time, which are ideal for the user.

In the aforementioned embodiment, the state determining device 10 is placed at a single location near the user. However, state determining devices 10 may be placed at a plurality of locations near the user. Specifically, the state determining devices 10 combine the numbers of body motions detected at a plurality of locations, uses the combined number, and may thereby obtain a more accurate result of determining the state of the user.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. A state determining device comprising:
a sensor to detect body motions of a test subject without physical contact; and
a processor to determine whether the test subject is asleep or awake based on numbers of body motions detected for predetermined time periods.

2. The state determining device according to claim 1, wherein
the processor calculates a state determination value that is an index of the determination, based on a first number of body motions detected for a first predetermined time period before a reference time and a second number of body motions detected for a second predetermined time period after the reference time, the reference time being a time for which the determination is made, and
the processor determines whether the test subject is asleep or awake based on a result of comparing the state determination value with a predetermined threshold value.

3. A state determination method executed by a state determining device including a sensor and a processor, the state determination method comprising:
detecting, by the sensor, body motions of a test subject without physical contact; and
determining, by the processor, whether the test subject is asleep or awake based on numbers of body motions detected for predetermined time periods.
